(19) United States
(12) Patent Application Publication
(10) Pub. No.: US 2010/0298777 A1
(43) Pub. Date: Nov. 25, 2010

(54) IV INFUSION SYSTEM

(75) Inventor: Vasu Nishtala, Snellville, GA (US)

Correspondence Address: KNOBBE MARTENS OLSON & BEAR LLP, 2040 MAIN STREET, FOURTEENTH FLOOR, IRVINE, CA, 92614, US

(73) Assignee: C.R. Bard, Inc., Covington, GA (US)

(21) Appl. No.: 12/675,740

(22) PCT Filed: Aug. 30, 2007
(86) PCT No.: PCT/US07/77302

§ 371 Date: Aug. 13, 2010

(51) Int. Cl. A61M 25/02

(52) U.S. Cl. 604/174

(57) ABSTRACT

A modular catheterization system includes a catheter retainer having an integral y-site lumen, and a secondary retainer for retaining an additional component in close proximity to the catheterization system. The y-site lumen comprises two fluid paths, where fluid flow through one of the paths is obstructed when no additional component is integrated into the catheterization system.

### BACKGROUND OF THE INVENTION

1. Field of the Invention

The present invention relates in general to a medical article anchoring system. In one mode, the present invention involves a catheterization system that interconnects an indwelling catheter with medical tubing and securely anchors the interconnection to a patient's skin.

2. Description of the Related Art

It is desirable to avoid removal and reinsertion of intravenous catheters. However, during the lifetime of an inserted intravenous catheter, it may become necessary to access and modify in some way a portion of an indwelling catheter. For instance, it may become necessary to provide access to the vasculature of a patient for delivery of removal of fluid from the bloodstream, or to monitor or measure some parameter. This may necessitate removal and reinsertion of the indwelling catheter, or may necessitate the insertion of a second intravenous catheter. As the insertion of an intravenous catheter carries with it some level of risk for the patient, it is desirable to minimize the need for such insertion whenever possible.

By providing a modular catheterization system, an indwelling catheterization system can be modified to include an additional component without disturbing the catheterization site or necessitating another catheter insertion. Such a catheterization system may be useful both when it is known in advance that the catheterization system will need to be modified, such as for scheduled monitoring of parameters, as well as when the need for a modification to the catheterization system is not known in advance.

### SUMMARY OF THE INVENTION

The systems and methods of the present invention have several features, no single one of which is solely responsible for its desirable attributes. Without limiting the scope of this invention as expressed by the claims which follow, its more prominent features will now be discussed briefly. After considering this discussion, and particularly after reading the section entitled “Detailed Description of the Preferred Embodiments,” one will understand how the features described herein provide advantages over traditional medical line securement systems.

An aspect is a modular catheterization system for placing a catheter in parallel flow communication with a medical line and a sensor. The system includes a first anchor pad and a housing that is supported by the first anchor pad and has a receiving area configured to receive at least a portion of the sensor. The system further includes a second anchor pad and a retainer that is supported by the second anchor pad. The retainer has a central body, a first tubular body, and a second tubular body. The central body has a proximal end configured to receive the catheter in a fluid-tight manner. The first tubular body and the central body form a first luminal path between the catheter and the medical line. The second tubular body and the central body form a second luminal path between the catheter and the sensor.

Another aspect is a catheterization system for co-locating a sensor and a catheter in close proximity on a patient. The system includes a first anchor and a first retainer that is supported by the first anchor. The first anchor has a receptacle, a manifold, a first conduit, and a second conduit. The receptacle is configured to receive at least a portion of the catheter. The first and second conduits are in fluidic communication with the manifold. A second anchor has a size and a shape so as to be located proximate to the first anchor. A second retainer is supported by the second anchor and has a receiving area configured to receive at least a portion of the sensor.

Another aspect is a modular catheterization system for allowing vascular access via an existing indwelling device, the system including a sensor housing disposed on a first anchor pad. The sensor housing includes a channel configured to retain a portion of a sensor. A catheter retainer is disposed on a second anchor pad. The catheter retainer includes a central body that has a first end. The first end is configured to be secured to and placed in fluid communication with a distal end of a catheter via an aperture extending through the first end. A first tubular body extends outward from the central body and comprises a second aperture. A first luminal path extends through the first tubular body from the second aperture to the first aperture. A second tubular body extends outward from the central body and includes a third aperture. A second luminal path extends through the second tubular body from the third aperture to the first aperture. An obstructing member is positioned along the second luminal path so as to inhibit fluid flow along the second luminal path without inhibiting fluid flow along the first luminal path. The system further includes a sensor having a body portion configured to be retained within the sensor housing and an outwardly extending member having a proximal end configured to enter the third aperture and interact with the obstructing member to permit passage of the proximal end beyond the obstructing member. The first and second anchor pads are configured to permit co-location of the sensor housing and catheter retainer.

### BRIEF DESCRIPTION OF THE DRAWINGS

FIG. 1 is a perspective view of a modular catheterization system that includes a catheter retainer secured to a component retainer according to a preferred embodiment of the present invention.

FIG. 2 is a perspective view of the catheter retainer component of the modular catheterization system illustrated in FIG. 1 securing a medical article to a patient.

FIG. 3 is a perspective view of an anchor pad from the catheter retainer of FIG. 2.

FIG. 4 is a perspective view of the catheter retainer of FIG. 2, shown apart from the anchor pad.

FIG. 5 is a top view of the catheter retainer of FIG. 2.

FIG. 6 is a front view of the catheter retainer of FIG. 2.

FIG. 7 is a back view of the catheter retainer of FIG. 2.

FIG. 8 is a left side view of the catheter retainer of FIG. 2.

FIG. 9 is a right side view of the catheter retainer of FIG. 2.

FIG. 10 is a cross-section of the catheter retainer of FIG. 2, taken along line 10-10 of FIG. 5.

FIG. 11 is a cross-section of the catheter retainer of FIG. 2, taken along line 11-11 of FIG. 5.

FIG. 12 is a bottom plan view of the catheter retainer of FIG. 2.

FIG. 13 is a perspective view of the catheter of FIG. 2.

FIG. 14 is a cross-section of the catheter of FIG. 2, taken along line 14-14 of FIG. 13.

FIG. 15 is an exploded assembly view of the catheter retainer and the medical article of FIG. 2.

FIG. 16 is a top plan view of the catheter retainer and the medical article of FIG. 2.

FIG. 17 is a cross-section taken along the line 17-17 of FIG. 16 and showing the medical article secured to the catheter retainer.

FIG. 18 is a perspective view of the assembly of FIG. 2 installed on the patient, and including a protective cap over one of the tubular bodies.

FIG. 19 is a perspective view of an embodiment of a component retainer and a retained illustrative sensor.

FIG. 20 is a perspective view of the anchor pad of the component retainer of FIG. 19.

FIG. 21 is a perspective view of the component retainer of FIG. 19 with the sensor removed.

FIG. 22 is a top plan view of the component retainer of FIG. 19.

FIG. 23 is a back view of the component retainer of FIG. 19.

FIG. 24 is a front view of the component retainer of FIG. 19.

FIG. 25 is a left side view of the component retainer of FIG. 19.

FIG. 26 is a right side view of the component retainer of FIG. 19.

FIG. 27 is a cross-section of the component retainer of FIG. 19, taken along the line 27-27 of FIG. 22.

FIG. 28 is a bottom plan view of the component retainer of FIG. 19.

FIG. 29 is a perspective view of the illustrative sensor of FIG. 19.

FIG. 30 is a cross-section of the sensor of FIG. 19, taken along the line 30-30 of FIG. 29

FIG. 31 is an exploded assembly view of the component retainer and sensor of FIG. 19.

FIG. 32 is a cross-section of the assembly of FIG. 19, taken along the line 32-32.

FIG. 33 is a perspective view of an alternate embodiment of a component retainer.

FIG. 34 is a top plan view of the component retainer of FIG. 33.

FIG. 35 is a left side view of the component retainer of FIG. 33.

FIG. 36 is a right side view of the component retainer of FIG. 33.

FIG. 37 is a back view of the component retainer of FIG. 33.

FIG. 38 is an exploded assembly view of the modular catheterization system of FIG. 1.

FIG. 39 is an enlarged perspective view of the modular catheterization system of FIG. 1.

FIG. 40 is an exploded assembly view of an alternate embodiment of the modular catheterization system.

FIG. 41 is a perspective view of the assembled modular catheterization system of FIG. 40.

FIG. 42 is a cross-section of the modular catheterization system of FIG. 41, taken along the line 42-42.

FIG. 43 is a cross-section of the modular catheterization system of FIG. 39, taken along the line 43-43

### DETAILED DESCRIPTION OF THE PREFERRED EMBODIMENTS

The present embodiment of the medical article anchoring system is disclosed in the context of a catheterization system, and in particular the context of a catheterization system including an integral Y-site lumen. The catheterization system can also include a catheter, a probe and probe housing, a tube extension set and/or dressing materials.

The principles of the present invention, however, are not limited the specific type of catheters or probes. Instead, it will be understood by one of skill in this art, in view of the present disclosure, that the anchoring system disclosed herein also can be successfully utilized in connection with other components to provide a modular system having a secondary fluid path which can be utilized when needed. The system may be used to allow vascular access via an existing indwelling device, so to achieve greater utility for patient monitoring, for example. In addition, it will be understood by one of skill in this art that the anchoring system disclosed herein also can be successfully utilized in connection with other types of medical articles, including other types of catheters, fluid drainage and delivery tubes. For example, but without limitation, the retainer disclosed herein can be configured to secure peripheral catheters, peripherally inserted central catheters, hemodialysis catheters, surgical drainage tubes, feeding tubes, chest tubes, nasogastric tubes, scopes, as well as electrical wires or cables connected to external or implanted electronic devices or sensors. One skilled in the art may also find additional applications for the devices and systems disclosed herein.

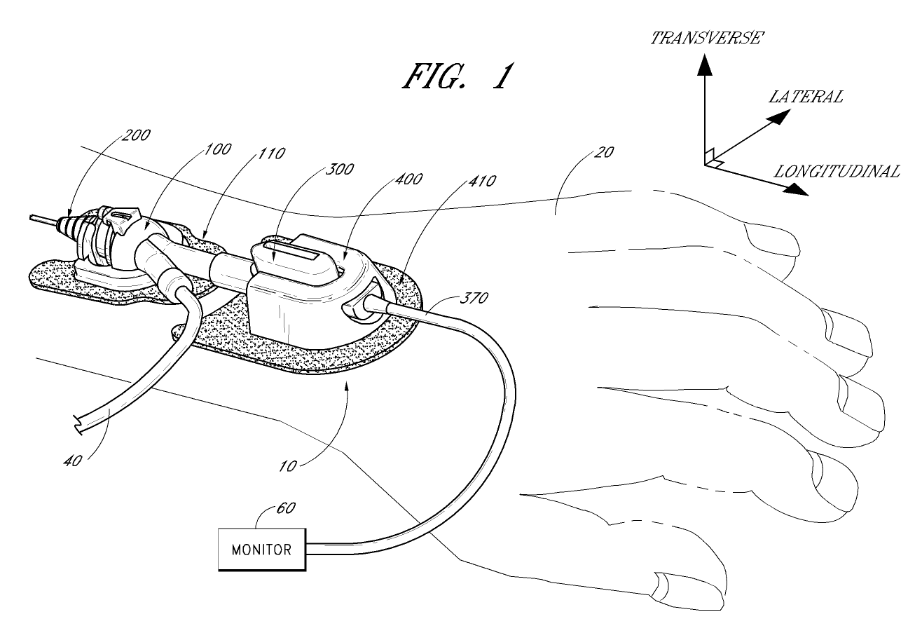

With reference now to a first embodiment, FIG. 1 illustrates in perspective view an assembled modular catheterization system 10. In particular, it can be seen that the illustrated modular catheterization system 10 comprises an embodiment of a catheter retainer 100 secured to the skin 20 of a patient by an adhesive layer on the underside of anchor pad 110. A medical article is retained and secured at the proximal end of the catheter retainer 100. In the illustrated embodiment the distal end 200 of the medical article is secured. The catheter retainer comprises at least two luminal paths extending in substantially distal directions, such that the catheter may be placed in fluid communication with tube 30, and also in fluid communication with sensor 300. In the illustrated embodiment the sensor 300 is an optrode sensor. It can be seen that sensor 300 is retained within a component retainer 400, which itself is secured to the skin 20 of the patient via an adhesive layer on the underside of anchor pad 410.

The anchor pads 110, 410 may be attached at any number of locations on a patient's body. For example, the modular catheterization system 10 may be located on the back of a patient's hand or arm, as shown. The modular catheterization system may be used for catheterization at other locations on the patient's body, e.g., on the medial side of the wrist in connection with catheterization of a radial artery, on the anterior or the posterior of the patient's torso in connection with epidural catheterization, or on or near the neck to provide access to large vessels such as the jugular or ceratoid veins/artery.

Further, as will be discussed in greater detail below, certain components of the modular catheterization system 10 need not be secured in place initially, but can be secured to the patient's body or integrated into the catheterization system when and if they are required. In particular, the modular catheterization system can be utilized initially with only the catheter retainer 100 securing the distal end 200 of the catheter, and without either the component retainer 400 or the sensor 300 secured in place. At a later time, these components may be integrated and secured in place, thereby avoiding additional puncture and providing access to blood within the body.

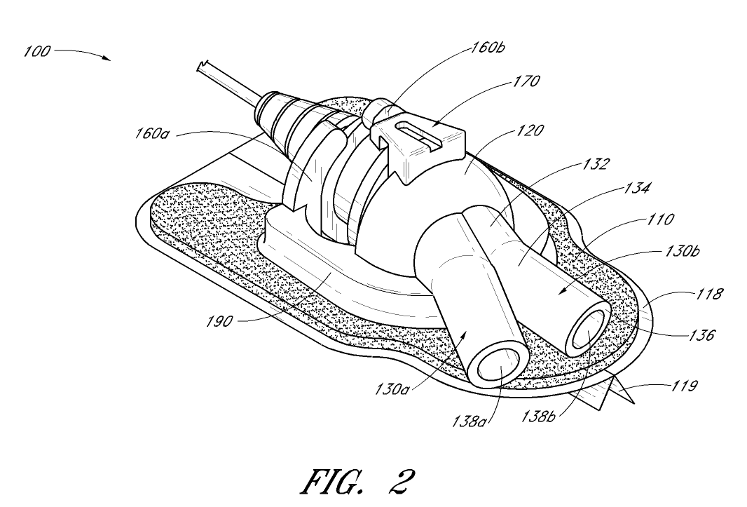

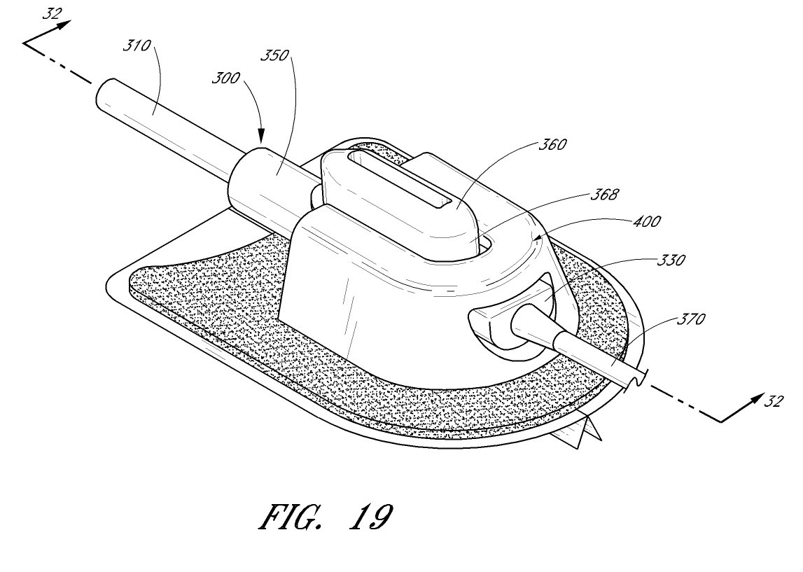

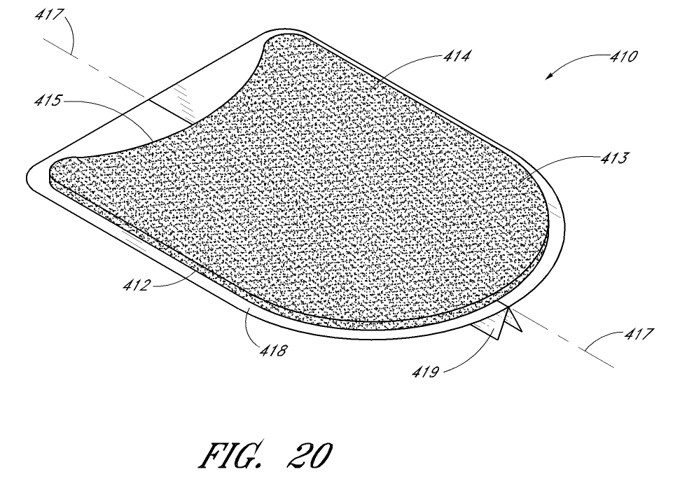

To assist in the description of the components of the catheterization system 10, and with respect to the present figure the following coordinate terms are used. A “longitudinal axis” is generally parallel to the central axis extending through the catheter retainer, the sensor, and the channel in the component retainer within which the sensor sits, as depicted in FIG. 1. A “lateral axis” is normal to the longitudinal axis and is generally parallel to the plane of the anchor pads 110 and 410. A “transverse axis” extends normal to both the longitudinal and lateral axes. In addition, as used herein, “the longitudinal direction” refers to a direction substantially parallel to the longitudinal axis; “the lateral direction” refers to a direction substantially parallel to the lateral axis; and “the transverse direction” refers to a direction substantially parallel to the transverse axis. The terms “proximal” and “distal”, which are used to describe the present catheterization system, are used consistently with the description of the exemplifying application. Thus, proximal and distal are used in reference to the catheter's insertion site 30 on the patient (see, e.g. FIGS. 18 and 19 showing an installed catheterization system). Also, the terms “top”, “bottom”, “upper”, and “lower” are used in the context of the orientation of the anchoring system illustrated in FIG. 2, and are not intended to imply a limitation to the orientation that the modular catheterization system 10 can assume on the patient.

### Anchor Pads

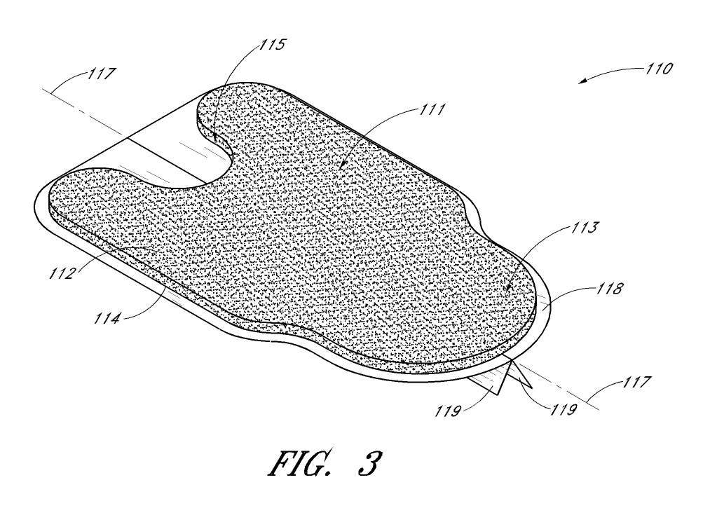

FIG. 3 illustrates a perspective view of the anchor pad 110 apart from the rest of the catheter retainer 100 (see FIG. 2 for a perspective view of the anchor pad shown in place with respect to the catheter retainer 100). The lower side of the pad 110 faces toward the skin of the patient, and is preferably covered with an adhesive suitable for attaching the anchor pad 110 to the skin of the patient. The entire surface, however, need not be covered. An upper surface 114 of the anchor pad 110 faces away from the skin of the patient and supports the catheter retainer 100 (see FIG. 2). The anchor pad 110 may comprise a central portion 111 with a substantially semicircular portion 113 extending from the distal end. A rounded notch 115 on the proximal end of the anchor pad 110 may provide additional support for the illustrated shape of the catheter retainer 100 without interfering with the insertion site. A medical provided may utilize the rounded notch 115 to align catheter retainer 100 with the insertion site in certain embodiments. Although only a single shape of the anchor pad 110 is illustrated in FIG. 3, those of skill in the art will recognize that a variety of shapes can be used and thus the anchor pad 110 is not limited to the illustrated shape.

The anchor pad 110 desirably comprises a laminate structure with an upper plastic, paper or foam layer (e.g., closed-cell polyethylene foam) and a lower adhesive layer. The lower adhesive layer constitutes a lower surface 112 of the anchor pad. The lower surface 112 desirably is a medical-grade adhesive and can be either diaphoretic or nondiaphoretic, depending upon the particular application. Such foam with an adhesive layer is available commercially from Avery Dennison of Painsville, Ohio. While not illustrated, the anchor pad 110 can include suture holes in addition to the adhesive layer to further secure the anchor pad to the patient's skin.

In other variations, a hydrocolloid adhesive or zinc oxide-based adhesive can advantageously be used upon the anchor pad 110 for attaching the anchor pad to the skin of the patient. The hydrocolloid or zinc oxide-based adhesive can be used either alone or in combination with another medical grade adhesive (e.g., in combination with the adhesive available from Avery Dennison). Hydrocolloid and zinc oxide-based adhesives have less of a tendency to excoriate the skin of a patient when removed. This can be particularly important for patients whose skin is more sensitive or fragile, such as neonates and those with a collagen deficiency or other skin related condition.

A surface of the upper foam layer constitutes an upper surface 114 of the anchor pad 110. The upper surface 114 can be roughened by corona-treating the foam with a low electric charge. The roughened or porous upper surface can improve the quality of the adhesive joint (which is described below) with the bottom surface of the catheter retainer 100.

A removable paper or plastic release liner 118 desirably covers the adhesive lower surface 112 before use. The liner 118 preferably resists tearing and desirably is divided into a plurality of pieces to ease attachment of the pad to a patient's skin.

The liner 118 comprises folded over portions to define pull tabs 119. The pull tabs 119 can be utilized to remove the paper or plastic release liner 118 from the adhesive lower surface 112 before use. A healthcare provider uses the pull tab 119 by grasping and pulling on it so that the liner 118 is separated from the lower surface 112. The pull tabs 119 overcomes any requirement that the healthcare provider pick at a corner edge or other segment of the liner 112 in order to separate the liner 112 from the adhesive layer.

The pull tabs 119 of course can be designed in a variety of configurations. For example, the pull tabs 119 can be located along a center line of the anchor pad 110 and oriented so as to face one another, as shown; or alternatively, one or more pull tabs can be located along any line of the anchor pad 110 in order to ease the application of the anchor pad 110 onto the patient's skin at a specific site. For example, an area of a patient's skin with an abrupt bend, such as at a joint, can require that the pull tab 119 be aligned toward one of the lateral ends of the anchor pad 110 rather than along the center line. In the embodiment illustrated in FIG. 3, the fold forming the pull tabs 119 is located along a centerline 117.

In another variation, the anchor pad 110 comprises a laminate structure with an upper woven layer and a lower adhesive layer. The upper layer can be polyester or other suitable polymer or textile materials. One particular suitable material is woven polyester available commercially under the name “Tricot” from Tyco. The lower adhesive layer constitutes the lower surface 112 of the anchor pad.

FIG. 20 illustrates a perspective view of the anchor pad 410 from FIG. 1 apart from the component retainer 400. The illustrated anchor pad comprises a semicircular distal end 413 and a scalloped proximal end 415 which may advantageously be contoured to accommodate the semicircular portion 113 on the distal end of the anchor pad 110. When configured in such a manner (as can be seen in, for example, FIG. 1), the catheter retainer 110 and the component retainer 400 may advantageously be positioned very close to one another, while still providing a desired level of stability, so as to minimize the area on the patient occupied by the modular catheterization system. The anchor pad 410 may have a structure similar to that of anchor pad 110. For example, the anchor pad 410 may have an adhesive layer on the lower surface 412 of the adhesive pad. The adhesive layer may be protected by a release liner 418. The release liner 418 may have two pull tabs 419 extending from either side of centerline 417. The upper surface 412 of the anchor pad 410 may be treated as described above with respect to anchor pad 410.

### Catheter and Catheter Retainer

Although a modular retainer system such as modular catheterization system 10 may be used to retain a wide variety of medical articles, the first embodiment will be described with respect to the illustrated catheter 200 and catheter retainer 100. It will be understood that in alternate embodiments, changes to the shape and type of medical article to be retained, and to the corresponding retainer, may be made without departing from the scope of the invention.

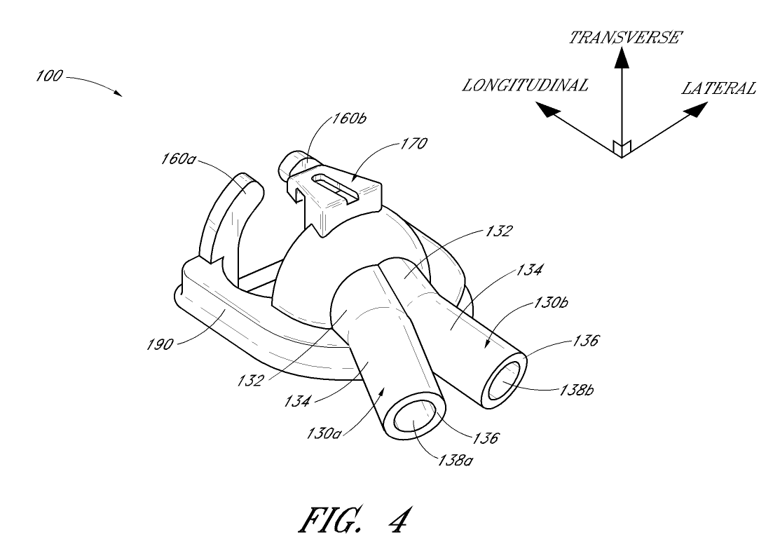

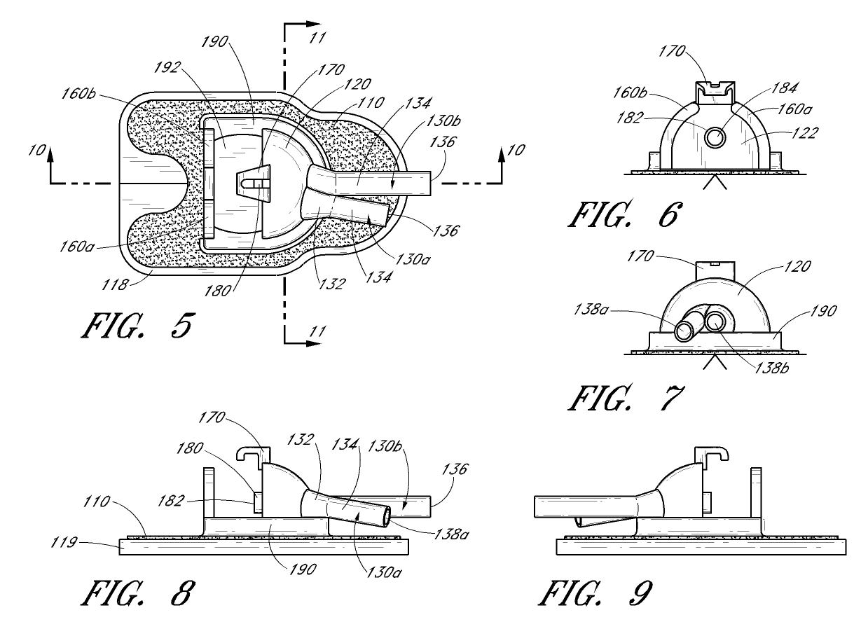

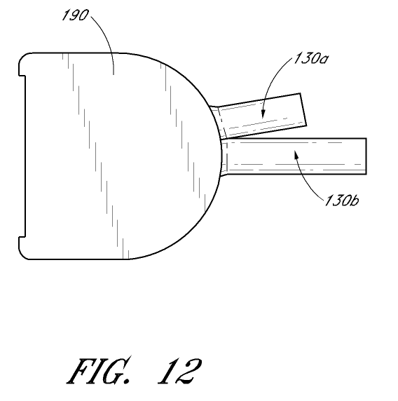

FIG. 2 illustrates a perspective view of a distal end 200 of the catheter retained within the catheter retainer 100. FIGS. 5-11 illustrate various views of the catheter retainer 100 with anchor pad 110 attached. FIGS. 4 and 12 illustrate a perspective view and a bottom plan view, respectively, of the catheter retainer 100 without the anchor pad 110.

As can be seen in FIG. 4, the catheter retainer 100 comprises a rounded central portion 120, having a partially spherical shape, and a substantially flat face 122 (see, e.g., FIG. 8) on the proximal side of the catheter retainer. Tubular bodies 130a and 130b extend in a substantially distal direction from the rounded distal side of the central portion 120. On the proximal side of the catheter retainer are securing features in the form of a pair of inwardly curving retaining tongs 160a and 160b, and overhanging clip 170. The securing features are configured to secure and retain the distal end of the catheter. Extending outward from the distal face of the central portion is a tubular body 180 configured to engage a corresponding opening on the distal end of the catheter so as to form a fluid tight seal with the catheter. Extending about the base of the catheter retainer is a footing 190.

As can best be seen in FIG. 5, tubular bodies 130a and 130b comprise frustro-conical tapering portions 132 adjacent to the rounded central portion 120, narrowing as they extend away from the central portion. Sections 134 having a constant cross-sectional diameter then extend from the distal end of the frustro-conical portions to the distal ends 136 of the tubular bodies. Circular apertures 138a, 138b are found at the distal ends 136 of the tubular bodies, and define the end of luminal paths extending through the catheter retainer. It can also be seen that the first tubular body 130a extends at an angle to the longitudinal axis when viewed from above, and the second tubular body 130b extends in a direction parallel to the longitudinal axis.

The catheter retainer 100 comprises an integral y-site lumen in which one of the luminal paths is initially occluded. In the illustrated embodiment, a first luminal path extends through the central portion from the aperture 184 at the proximal end 182 of tubular body 180 to the aperture 138a at the distal end 136 of tubular body 130a. This first luminal path is unoccluded by any features within the catheter retainer. When the catheter is placed in fluid communication with the aperture 184, a fluid path between the catheter and the distal end of the first tubular body 130a is formed. The fluid path runs through the catheter retainer via lumen 133a extending through the first tubular body 130a (see FIG. 11). An IV bag or other medical article can be placed in fluid communication with the first tubular body 130a via tubing such as tubing 40 (see, e.g. FIG. 1), which in the illustrated embodiment can be stretched over the tubular body so as to provide the desired fluid tight seal.

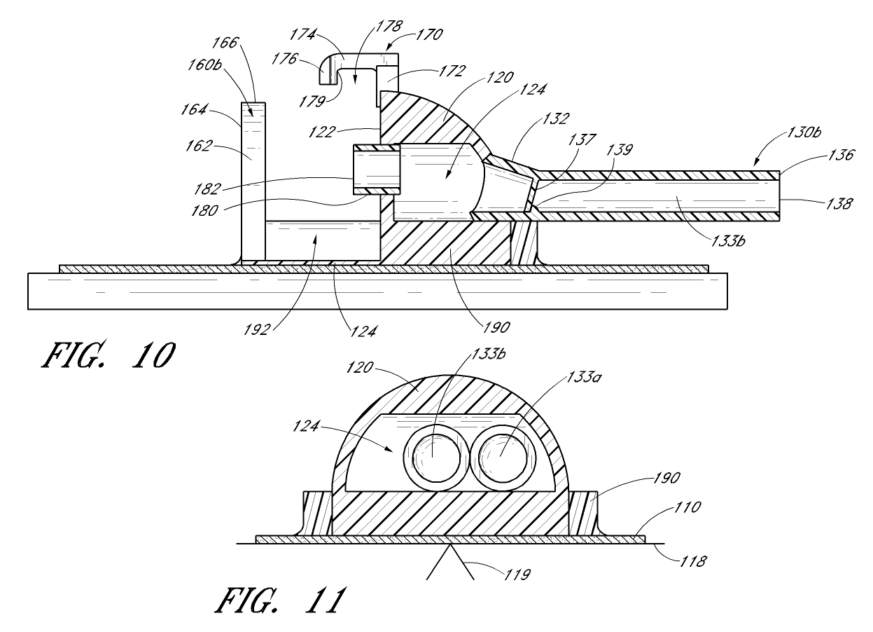

As can be seen in FIG. 10, which is a cross-section taken along the line 10-10 of FIG. 5, a second luminal path extends between the aperture 184 at the proximal end of the tubular body 180 and the aperture 138b at the distal end of the second tubular body 130b. The second luminal path is preferably occluded at a point within second tubular body 130b via a movable septum 137. The septum may have a flap biased in the distal direction against a stop 139 within the interior of the second tubular body 130b. Although the illustrated embodiment comprises a movable flap, it will be understood that the second luminal path may be occluded through other means, such as via a pierceable septum, or a septum having a slit extending therethrough which is biased to remain closed. Thus, when the catheter retainer is placed in fluid communication with a catheter, fluid is inhibited from flowing through the second luminal path via the septum 137.

As will be described in greater detail below, the flap of septum 137 may be located at the transition from the frustro-conical portion 132 of second tubular body 130b to the constant diameter section 134. Because the lumen 133 widens at this transition when moving in a proximal direction, the septum 137 may be pushed upward into the upper portion of the lumen without further narrowing the effective width of the lumen 133.

The second tubular body 130b thus serves as an access port, which provides access to the fluid flow through the catheter retainer. In certain embodiments, the access port may provide access to venous blood flowing through the catheter retainer. In the illustrated embodiment, the access port is self-sealing due to the bias of the movable septum.

Still with respect to FIG. 10, it can be seen that the tubular body 180 extending through the distal wall of the central portion 120 may extend partially into the interior cavity 124 of the central portion, so as to provide additional stability for the tubular portion. It can also be seen that the tubular body 180 may be vertically offset from the second tubular body 130b, such that a member inserted into the interior cavity of the central portion may be inhibited from extending into the tubular body 180, as will be discussed in greater detail below. Furthermore, although the interior cavity is shown as having substantially horizontal upper and lower walls, and side walls that conform to the shape of the exterior of the central portion, the size of the interior cavity may be increased or decreased as desired by altering the thicknesses of the walls of the central portion.

As can best be seen in FIGS. 4, 5, and 8, the catheter retainer 100 comprises securement features configured to retain and secure in place the distal end of a catheter. When secured, the catheter retainer 100 provides a fluid tight seal between the catheter and the interior luminal paths extending through the catheter retainer. In the illustrated embodiment, the catheter retainer 100 comprises resilient tong members 160a, 160b. The tong members extend transversely upward from the base 124 of the catheter retainer and curve laterally inward above the center of the aperture 184 extending through the tubular body 180. The tong members 160a,b comprise interior surfaces 162 and proximal surfaces 164. The interior surfaces 162 are disposed along the inward-facing side of the tong members. The proximal surfaces 164 are disposed along the proximal faces of the tong members. The radii of curvature of the inwardly curving interior surfaces 162 preferably matches or approximates the radius of curvature of the section of the distal end of the catheter to be retained, as will be described in greater detail below. The upper surfaces 166 of the tong members 160a,b are preferably rounded to facilitate placement of the retained distal end of the catheter.

An additional securement feature is provided by overhanging resilient clip 170. The clip 170 comprises a first vertical portion 172 extending transversely upwards from the highest point of the central portion 120, a proximally extending horizontal overhanging portion 174, and a clip portion 176 which extends transversely downwardly from the proximal edge of the overhanging portion 174. The interior surfaces of the portions 172, 174, 176 define a notched retaining area 178. The interior surface of the clip portion 176 facing the proximal face 122 of the central portion serves as an interior retaining surface 179.

Footing 190 extends underneath and about the edges of the catheter retainer 100. It can be seen that the base of the footing 190 tapers outward so as to provide additional stability for the catheter retainer. It can also be seen that footing 190 comprises a rounded depression 192 between tong members 160a,b and the proximal face of the central portion 120, which permits entry of the retained distal end of the catheter. The proximal edge of the rounded depression curves transversely inward to provide additional support for tong members 160a,b, as can best be seen in FIG. 4. The rounded nature of the depression 192 facilitates detachment of the catheter from the catheter retainer by allowing a medical professional to reach to the side and beneath the retained portion of the catheter so as to pull it upwards, as will be discussed in greater detail below.

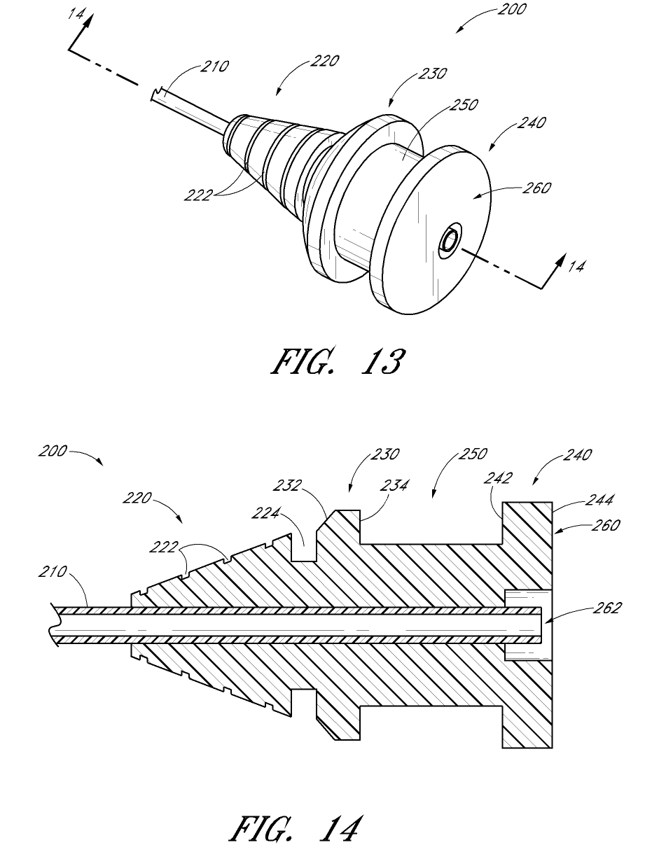

FIGS. 13 and 14 illustrate a perspective view and a cross-section, respectively, of the distal end 200 of the catheter to be retained within the catheter retainer 100. It can be seen that the catheter comprises tubing 210 extending in a proximal direction towards the insertion site, and that the distal end 200 comprises a structure configured to be retained by the catheter retainer. In particular, the distal end 200 comprises a conical portion 220 tapering outward in a distal direction. The conical portion 200 may comprise grooves 222 or other scoring so as to facilitate gripping of the conical portion by a medical professional.

Distal of the conical portion 220 are a proximal radially extending section 230 and a distal radially extending section 240, separated by a narrower cylindrical neck portion 250. In the illustrated embodiment, the distal edge of the proximal radially extending portion 230 may comprise a bevel 232, and the proximal radially extending portion 230 may be separated from the conical portion by a radial notch 224, in order to further facilitate gripping of the distal end 200 of the catheter. The distal face 260 of the distal end 200 comprises a cylindrical indentation 262 having an interior diameter substantially equal to the outer diameter of tubular body 180. As can be seen, the tubing 210 may extend at least partway into the cylindrical indentation 262, so long as the outer diameter of the tubing 210 is less than or equal to the inner diameter of tubular body 182.

The distal face of the proximal radially extending section 230 is substantially vertical, and serves as a proximal abutment surface 234. Similarly, the proximal face of the distal radially extending section 240 is substantially vertical and serves as a first distal abutment surface 242, and the distal face of section 240 is substantially vertical and serves as a second distal abutment surface 244. As can be seen in FIG. 14, the diameters of the distal and proximal radially extending portions 230 and 240 may be substantially equal.

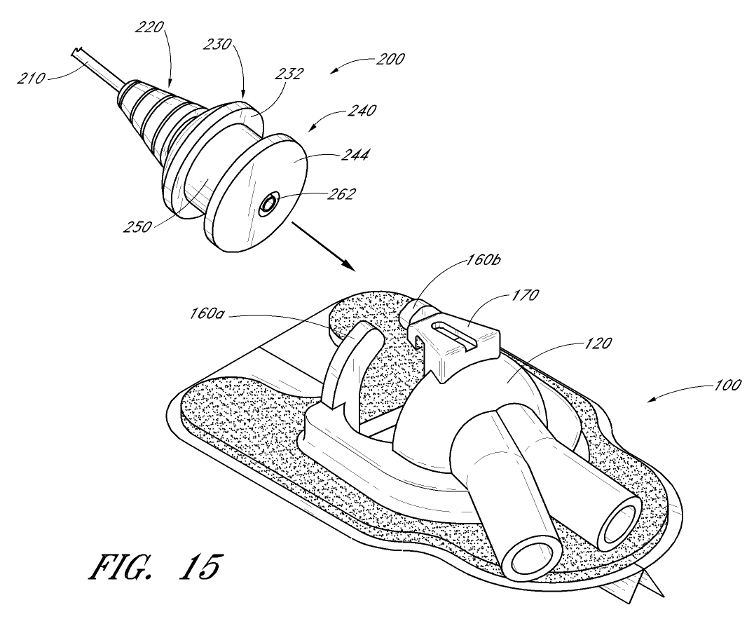

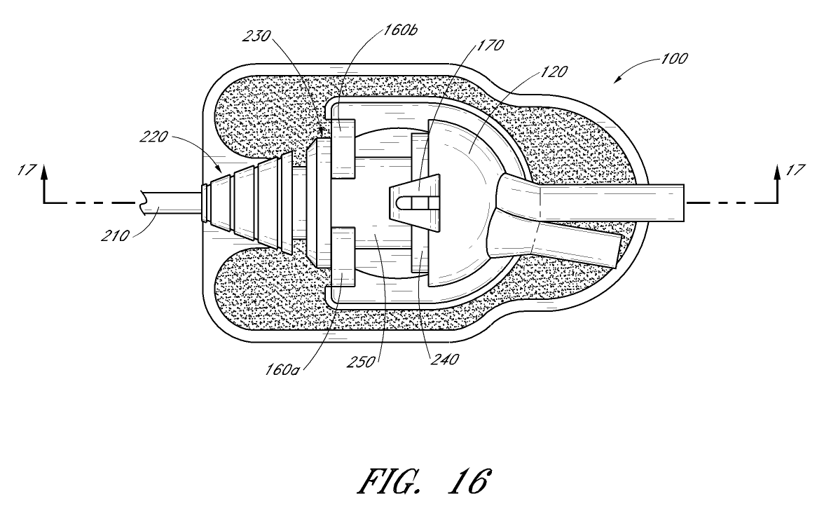

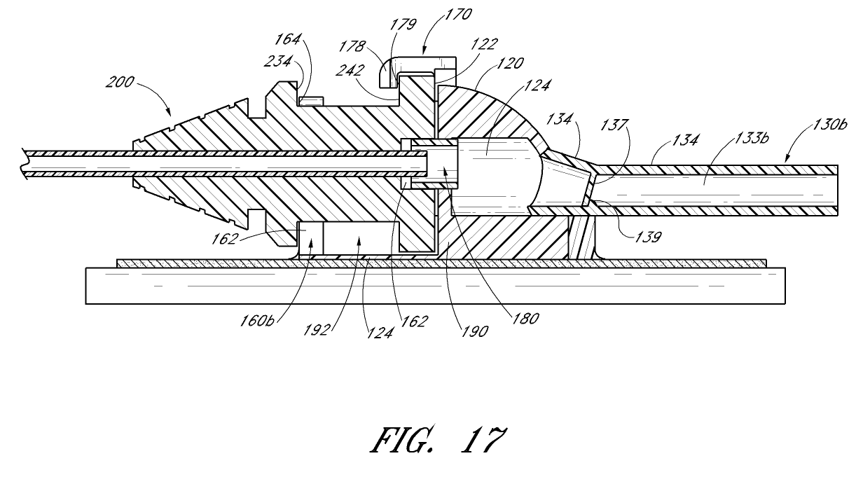

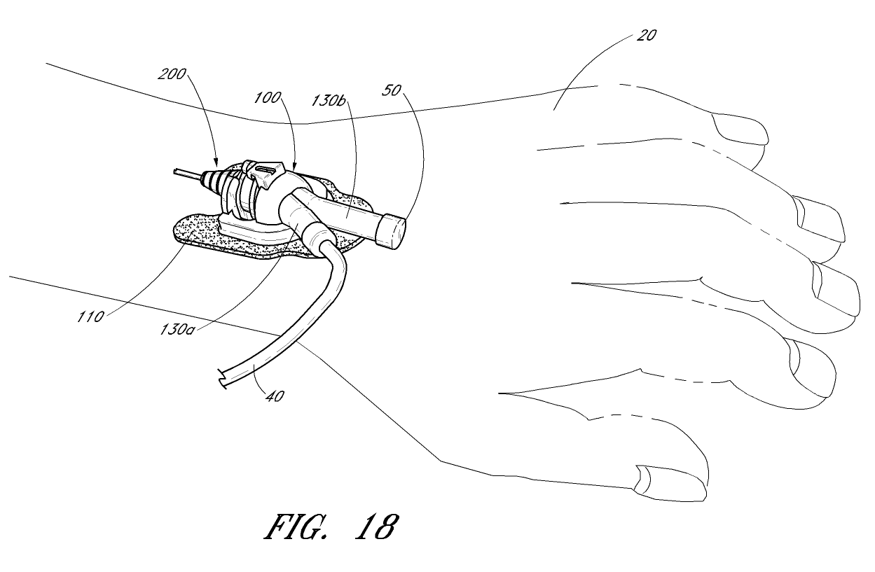

FIGS. 15-17 illustrate securement of the distal end 200 of the catheter within the catheter retainer 100. FIG. 15 is an exploded assembly view of the catheter retainer 100 and the medical article of FIG. 2. FIG. 16 is a top plan view of the catheter retainer 100 and the medical article of FIG. 2. FIG. 17 is a cross-section taken along the line 17-17 of FIG. 16 and showing the medical article secured to the catheter retainer 100.

The medical professional may insert the neck 250 of the catheter between the tongs 160a,b of the catheter retainer 100. In the illustrated embodiment, the vertical portions of the tongs are spaced apart by a distance substantially equal to the diameter of the neck portion, and the radius of curvature of the inwardly curving interior surfaces 162 of the upper portion of the tongs is substantially equal to that of the exterior of the neck portion. In addition, depth of the rounded depression 192 within the footing 190 is such that the tubular body 180 of the catheter retainer is vertically aligned with the cylindrical indentation 262 on the distal face of the catheter. The overhanging clip 170 may then be deformed upwards by the medical professional, and the catheter slid in a proximal direction relative to the catheter retainer, so that the tubular body 180 engages the cylindrical indentation, forming a fluid tight seal between the exterior surface of the tubular body and the interior curved surface of the retainer. The overhanging clip is then released and moves down to secure the distal radially extending portion 240 of the catheter.

Thus, it can be seen in FIG. 17 that longitudinal motion in the distal direction is inhibited by the distal face 260 of the catheter end abutting the proximal face of the central portion 120, and that longitudinal motion in the proximal direction is inhibited by the interior retaining surface 179 of the clip 170. Lateral and transverse motion is inhibited by the tongs 160a,b, the base 124 of the depression 192 in the footing 190, and the interaction between the exterior of tubular body 180 and the interior of cylindrical indentation 262. It can also be seen that the longitudinal length of the neck portion 250 is preferably at least the length of the distance between the proximal faces of tongs 160a,b and the interior retaining surface 179, so that the catheter can be moved sufficiently distal to allow the clip 170 to engage the distal radially extending member 240.

FIG. 18 illustrates a modular catheterization system in which only the catheter retainer 100 has been secured to the patient, and in which the distal end 200 of the catheter has been secured to the retainer. In the illustrated embodiment, although fluid flow out of the tubular body 130b is occluded by the septum within the tubular body, a protective cap 50 has been provided over the distal end 136 of the tubular body 130b to ensure that the portion of the tubular body extending distally beyond the septum 137 remains sterile. Thus, the modular catheterization system provides fluid flow through the interior of the catheter retainer via tubular body 130a and tubing 40, which may in some embodiments may be secured to port 130a by suitable connection features, such as a Luer lock and/or locking threads (not shown). In addition, however, the modular catheterization system provides the ability to install, at a later date, an additional component with access to the fluid flow through the interior of the catheter retainer. This additional component may be installed without disturbing the catheterization site or the already installed components. An embodiment of such an additional component is discussed below.

### Component Retainer and Sensor

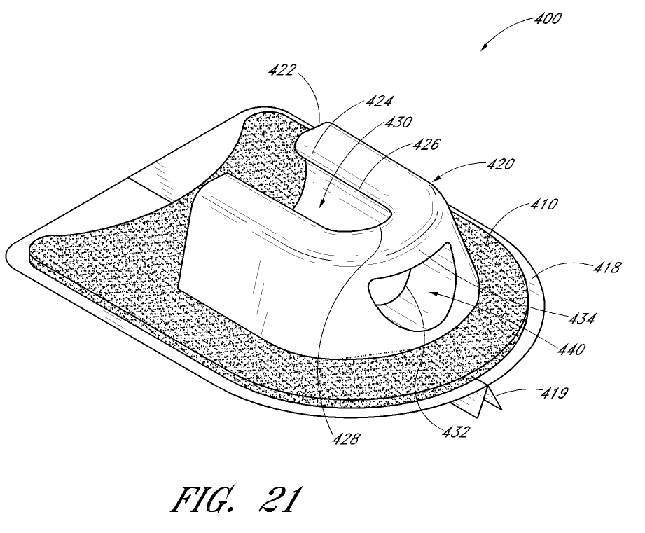

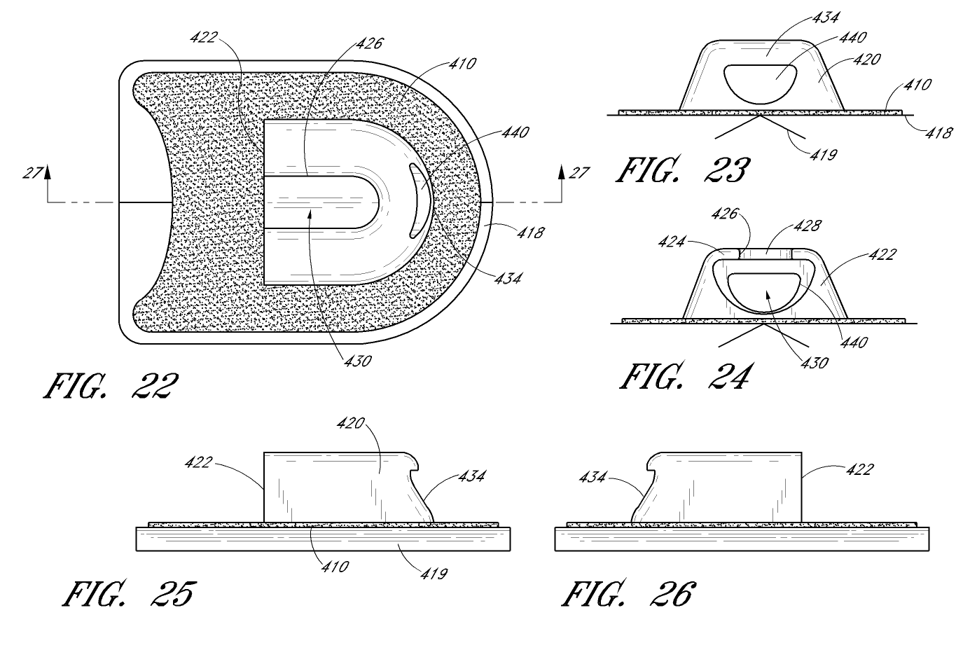

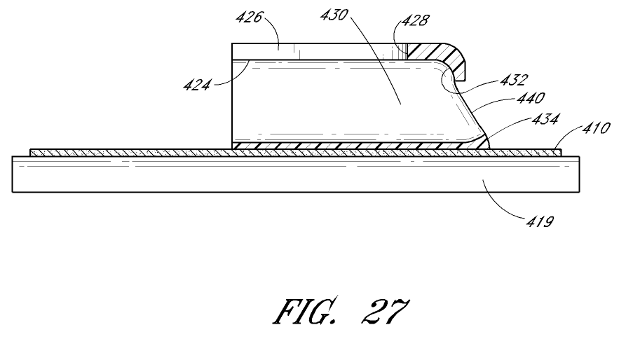

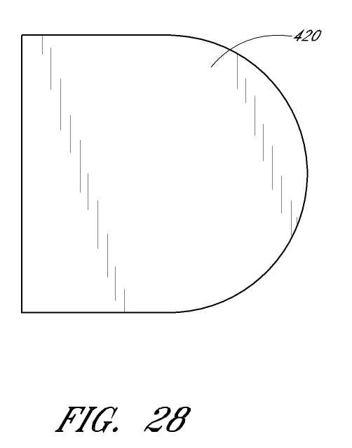

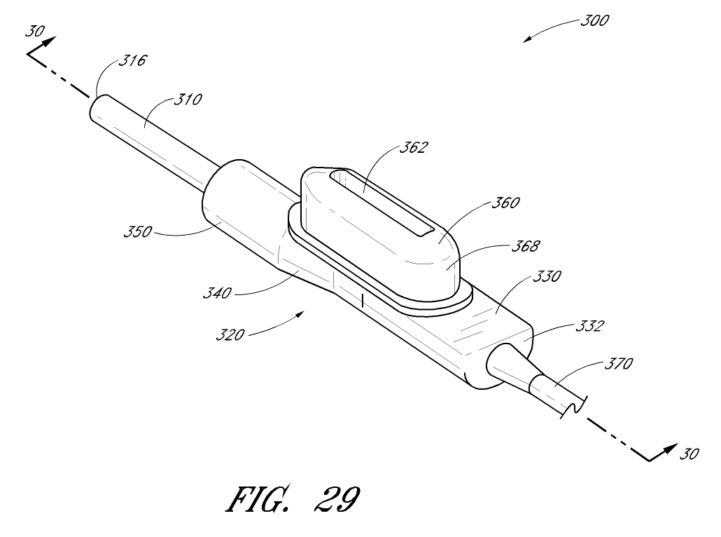

FIGS. 21-28 illustrate various views of a component retainer 400 configured to retain an additional component to be placed in fluid communication with the fluid path running through the interior of the catheter retainer 100. It will be understood, however, that the particular size and shape of the component retainer suitable for use with a given modular catheterization system will depend highly on the size and shape of the component to be retained, as well as any necessary connections with exterior system components.

It can be seen that the component retainer 400, which in the illustrated embodiment is a retainer for a probe 300, comprises a substantially U-shaped retaining structure 420 disposed on an anchor pad 410. An adhesive desirably attaches the retaining structure 420 to the anchor pad 410. Alternatively, the retaining structure 420 may be attached to the anchor pad 410 by non-adhesive means (e.g., embedding or otherwise weaving the retaining structure 420 into the anchor pad 410).

The U-shaped retaining structure 420 comprises a substantially U-shaped channel 430 extending in a longitudinal direction from the proximal end 422 of the retaining structure, the channel terminating at the distal end 432 of the channel at a rounded portion having a curvature which may match the curvature of the distal end 434 of the retaining structure 420. It can also be seen most clearly in FIG. 24 that the retaining structure comprises an overhanging lip 424 having an underside which is substantially horizontal near the center of the retaining structure 420. A U-shaped cutout 426 in the overhanging lip 424 extends from the proximal end of the channel 430 and terminates in a rounded distal edge 428 having a substantially vertical sidewall. It can be seen in FIG. 24 that the outer surface 450 of the retaining structure 420 tapers inward when moving upward in a longitudinal direction away from the base 460 of the retaining structure.

At the distal end 432 of the channel 430, an aperture 440 extends between the distal end of the channel and the distal end 424 of the retaining structure 420. It can best be seen in FIG. 24 that the aperture 440 has a cross-sectional shape which is similar in shape to that of the channel 430 but slightly smaller in size. Thus, when the article to be retained within retaining structure 420 has a portion having a cross-sectional shape substantially equal to that of the channel, the portion of the distal end 432 of the channel surrounding aperture 440 will serve as an abutment surface.

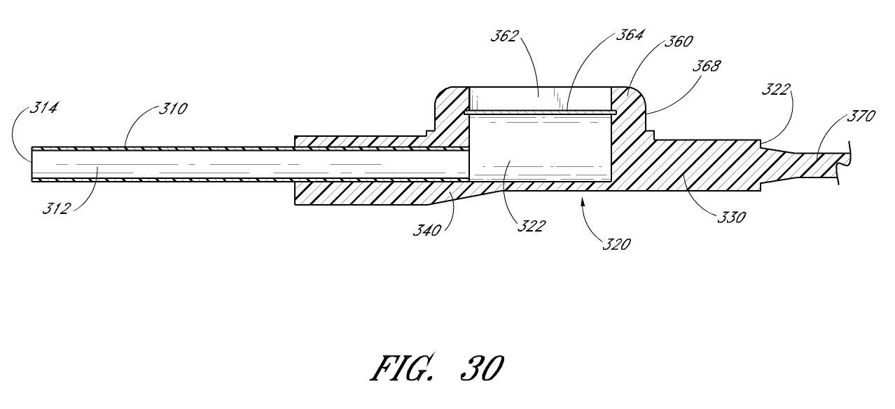

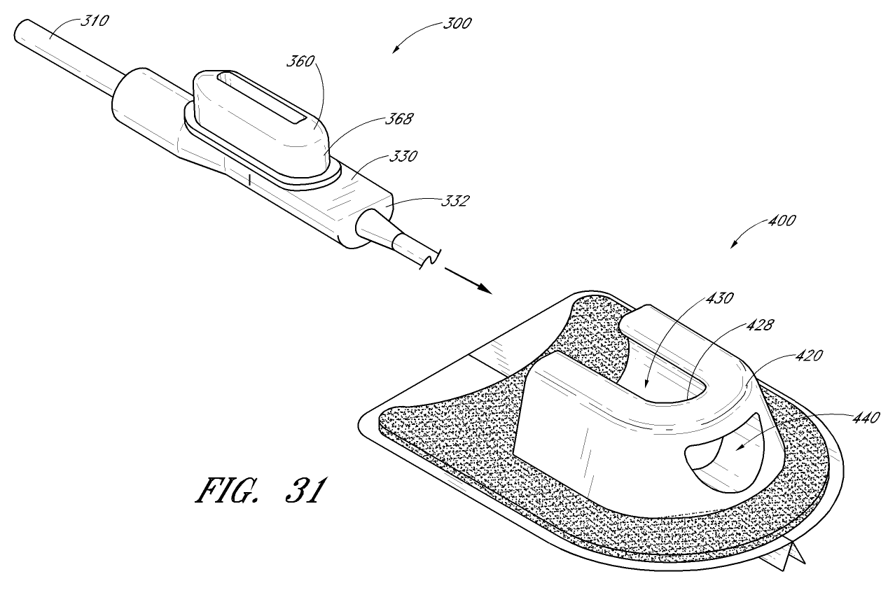

FIGS. 29 and 30 illustrate an exemplary sensor 300 to be retained within component retainer 400. In the illustrated embodiment, the sensor 300 comprises an optrode probe, but it will be understood that a wide variety of devices may be integrated into the modular catheterization system via tubular body 130b. The sensor 300 comprises a substantially rigid tubular body 310 extending in a proximal direction away from the central body 320 of the probe. It can be seen in FIG. 30 that the tubular body 310 comprises an interior lumen 312 extending from aperture 314 at distal end 316 of the probe into the central body 320 of the probe.

The central body 320 of the probe comprises an interior reservoir 322 into which blood flowing from catheter 200 may flow once the probe is placed in fluid communication with the fluid path. The exterior of central body 320 comprises a distal portion 330 which is shaped to fit within U-shaped channel 430 of the component retainer 400. In particular, it can be seen that the edges of the distal end 332 of the probe may abut the distal end of the channel 430.

A central transition portion 340 between the distal portion 330 and the proximal portion 350 has a cross-section which widens in the proximal direction to a point at which longitudinal translation into the channel 430 of the retainer 400 is inhibited. The wider distal portion 350 is thus inhibited from moving beyond the proximal end of the channel 430.

A rounded upper portion 360 extends transversely upward from the central body 320 of the probe. In certain embodiments, the upper portion may comprise a window into the probe, to ensure that blood is contained within the reservoir 322. In other embodiments, the upper portion 360 comprises a plastic interface mounted on the sensor shaft and configured to be retained within the retainer 400. In other embodiments, the upper portion may comprise an electronic display or other indicia of the function of the probe. In the illustrated embodiment, the upper portion comprises an aperture 362 separated from the reservoir by transparent or translucent layer 364. The rounded outer surface 366 of the distal end 368 of the upper portion has a cross-sectional shape when viewed from above which is configured to fit within the U-shaped cutout 426 above the channel 430.

Extending distally from the distal end of the probe 300 is cable 370. In the illustrated embodiment, the cable 370 may comprise the fiber optics which guide the light across a sample of blood within the reservoir. As most clearly shown in FIGS. 1 and 29, the cable 370 may connect to a monitor 60 or other medical device. The monitor 60 receives data from the probe 300 via the cable 370. In other embodiments, the cable 370 may comprise only electrical signals which control a self-contained optrode probe. In further embodiments in which another device is retained within component retainer 400, the cabling may comprise a further luminal path so that a device distal of the component retainer may be placed in fluid communication with the modular catheterization system.

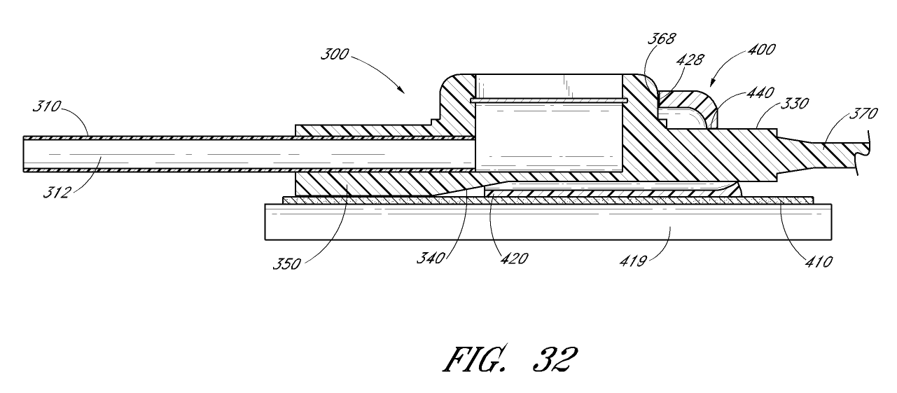

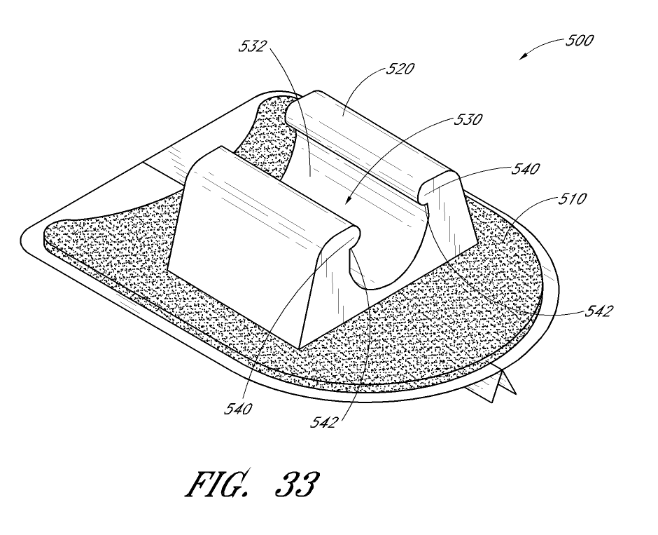

FIGS. 19, 31 and 32 illustrate the securement and retaining of the probe 300 within the component retainer 400. In the illustrated embodiment, it can be seen that longitudinal translation of the probe in the distal direction is inhibited by the outer surface 366 of the distal end of the upper portion, which abuts the rounded distal edge 428 of the U-shaped cutout in the upper surface of the retainer. Longitudinal translation is further inhibited by the widening of the transition section 340 of the probe, the lower surface of which abuts the lower surface of the proximal end of the retainer. Lateral and transverse translation of the probe is inhibited by the interaction of the outer surfaces of the distal section 330 of the probe which abut the interior surfaces of the aperture 440.

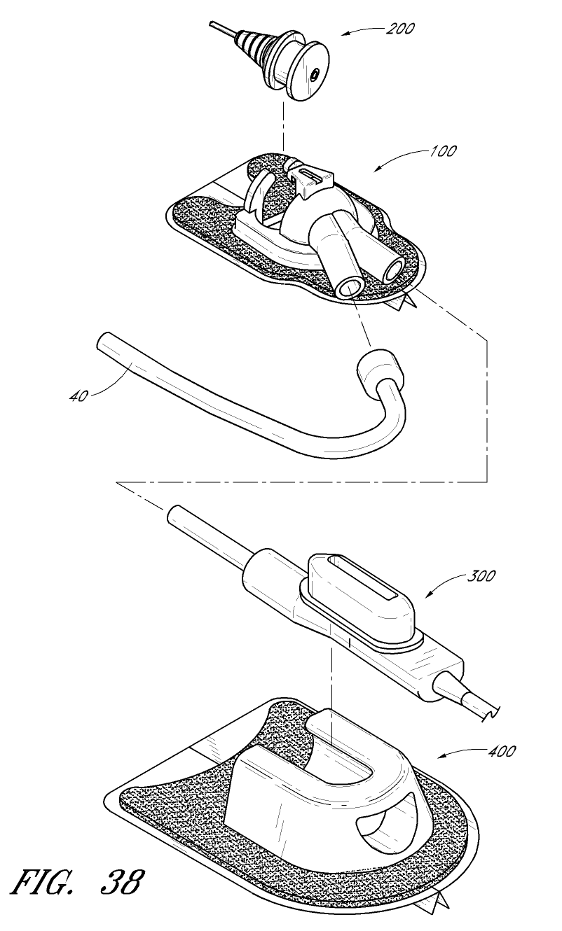

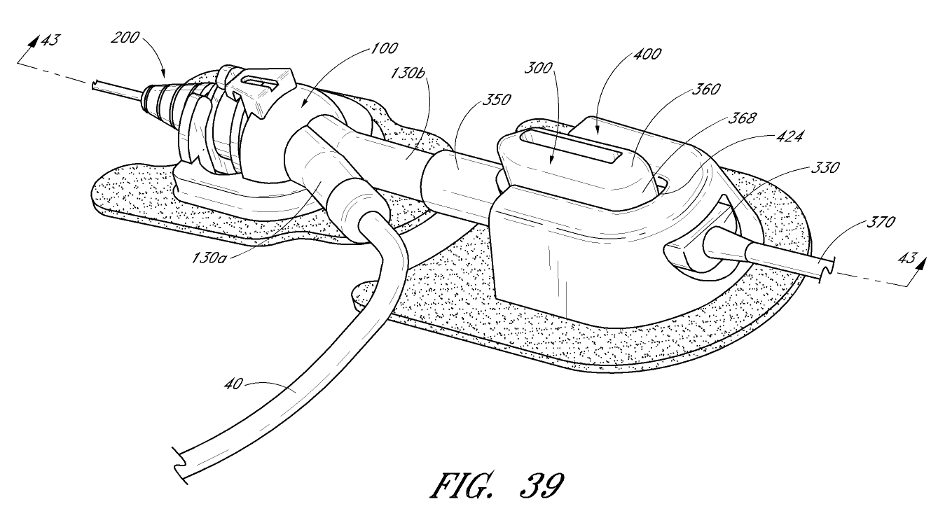

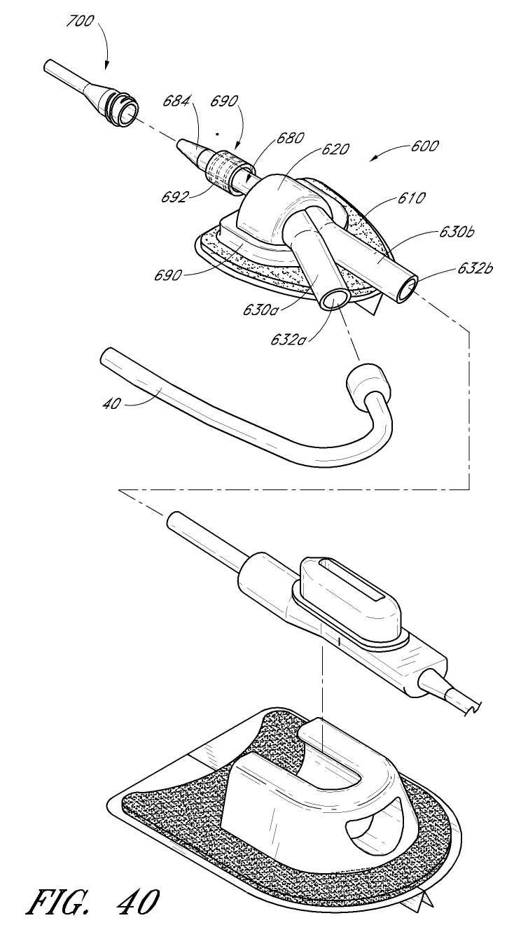

FIGS. 38, 39, and 43 illustrate the integration of the sensor and component retainer into the modular catheterization system. In particular, it can be seen in FIG. 43 that the rigid tubular body 310 has been inserted into the interior of tubular body 130b, and extended in a proximal direction so that the septum flap 1_{—}8 has been pushed inward and out of the way into the widening frustro-conical portion 134 of tubular body 130b. Thus, it can be seen that the probe 300 has been placed in fluid communication with the catheter tubing via the second luminal path extending through the catheter housing 100. It can also be seen that the vertical offset of the tubular bodies 180 and 130b prevents occlusion of or other interference with tubular body 180 if tubular body 310 is inserted too far in the distal direction via tubular body 130b. Thus, it can be ensured that the first luminal path through the catheter housing to tubular body 130a remains open.

### Alternate Embodiments

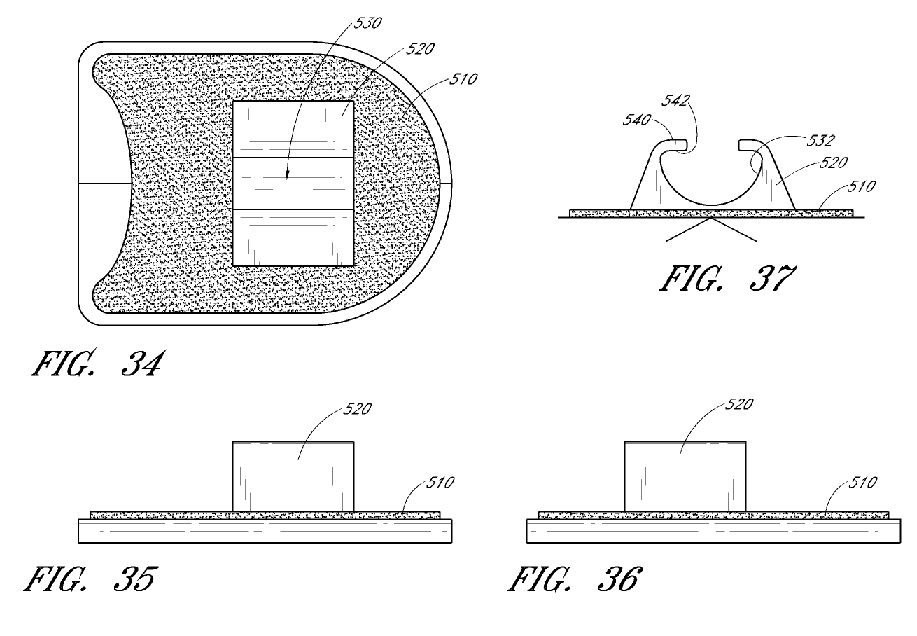

As discussed above, a wide variety of variations may be made to the design of the various components so as to accommodate a variety of components or connector designs. FIGS. 33-36 illustrate various views of an alternate component retainer. The alternate component retainer 500 comprises a retaining structure 520 seated on an anchor pad 510 similar to the anchor pad 410 previously discussed. A U-shaped channel 530 having a substantially constant cross-sectional shape extends in a longitudinal direction through the retaining structure 520. Overhanging lips 540 extend longitudinally above either side of the U-shaped channel, providing abutment surfaces 542 on the underside of the lips 540 to inhibit upward transverse translation of a retained article. Inhibition of lateral translation is provided by the side walls 432 of the U-shaped channel 430, and inhibition of longitudinal translation may be provided, e.g., via friction between the walls of the U-shaped channel and the retained article, or through the use of a retained article having a widening transition portion which will abut the proximal end of the retaining structure 520. An adhesive spot may also be utilized between the walls of the channel and the medical article to inhibit longitudinal movement of the medical article through the retainer.

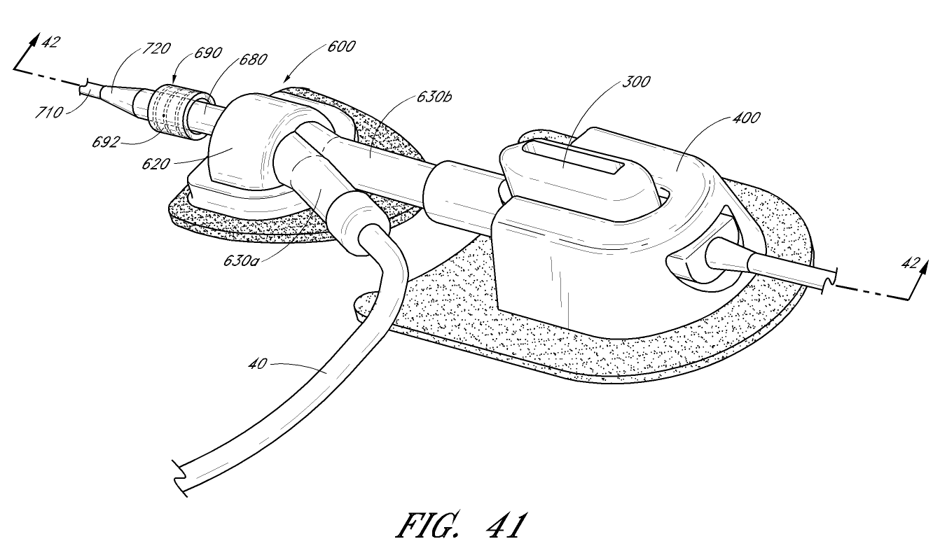

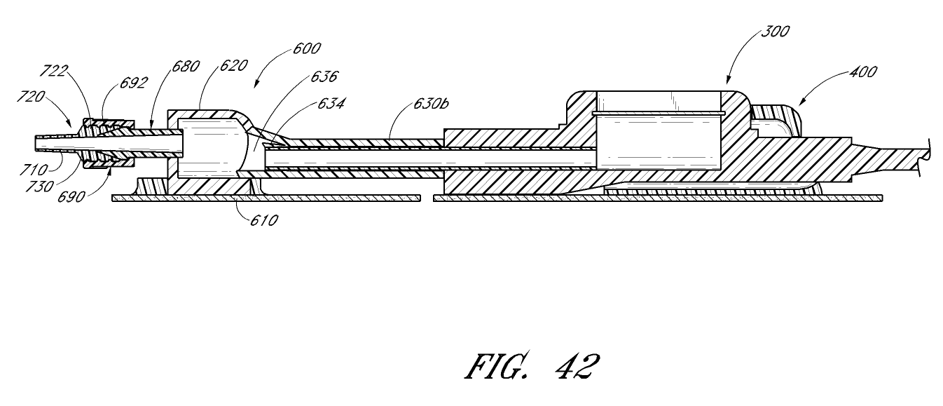

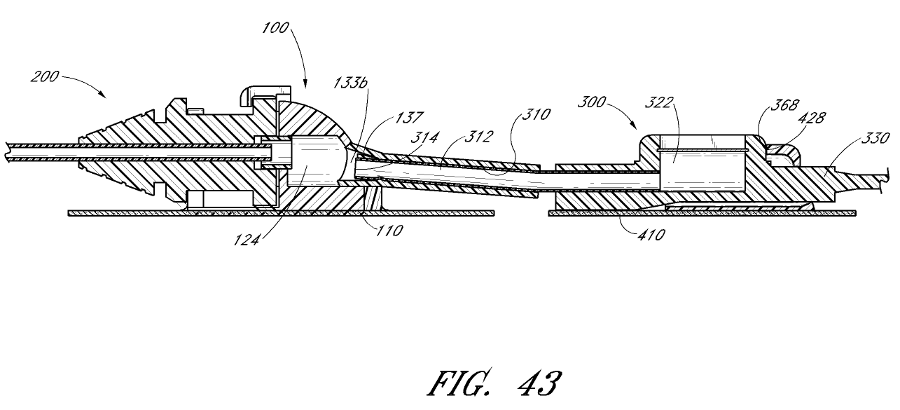

FIGS. 40-42 illustrate an alternate embodiment of a catheter retainer 600 and the distal end 700 of a catheter, wherein a connection between the catheter and the catheter retainer is provided via a spin nut disposed upon the catheter retainer. In particular, as can best be seen in FIGS. 40 and 42, the catheter retainer 600 comprises a central portion 620 disposed upon an anchor pad 610 which may be similar in structure to the anchor pad 110 previously discussed. Extending in the distal direction from the distal side of central portion 620 are tubular bodies 630a and 630b. The tubular bodies 630a and 630b are oriented at an angle to one another. Extending from the proximal end of the central portion 620 is a tubular member 680. As discussed previously with respect to catheter retainer 100, catheter retainer 600 comprises an integral y-site lumen having a first unoccluded path which extends from an aperture in the proximal tip 682 of the tubular member 680 to aperture 632a at the distal end of tubular body 630a. A second luminal path extends from the proximal tip 682 to the aperture 632b at the distal end of the second tubular body 630b. As previously discussed, this second luminal path is initially occluded by septum 634, which includes a movable flap biased to remain in a closed position, sealing the lumen 636 extending through the interior of the second tubular body 630b.

It can also be seen that the tubular member 680 comprises a frustro-conical narrowing section 684 at the proximal end. Spin nut 690, which is substantially cylindrical in shape and has interior threading 692 on its interior surface (shown in phantom in FIG. 40), is disposed upon the tubular member 680 and is movable in a longitudinal direction and freely rotatable about the tubular member. Movement in the distal direction is ultimately inhibited by the presence of the housing 620, and proximal movement beyond a desired position on the tubular member 680 may be inhibited, as well, such as, for example, by an inwardly extending member (not shown) on the interior of the distal end of the spin nut 690, a radially extending ridge on the tubular member 680, or some combination of the two.

The distal end 700 of the catheter comprises a distal section 710 with a substantial cylindrical outer surface and an aperture 720 configured to accept the frustro-conical tip 684 of tubular member. The catheter tip comprises an interior surface corresponding to the tapered shape of the exterior of the tip 684 of tubular member 680, so as to provide a fluid tight seal and to inhibit further proximal translation of the tubular member into the catheter tip. The catheter tip further comprises a narrowing transition portion 730 on the proximal side of the distal section 710, and tubing 740 extending from the proximal edge of transition portion 730 and extending towards the insertion site. It can also be seen that the distal section 710 comprises exterior threading 712 corresponding to the interior threading 692 within spin nut 690.

Thus, in one embodiment, the shape of the proximal end 682 of the tubular member 680, in conjunction with the spin nut 690, permits a catheter to be releasably secured to the catheter retainer 600. The spin nut 690 may first be moved to a distal position on the tubular member 680. The distal end 700 of the catheter is secured to the retainer by inserting the proximal end 682 of the tubular member 680 into the aperture 720 at the distal end of the catheter. The catheter is thus placed in fluid communication with the catheter retainer 600 and held in place initially by friction between the exterior surface of the frustro-conical portion 684 of the tubular member 680, and the interior surface of the distal section 710 of the catheter or adapter, forming a fluid tight seal. The spin nut 690 may then be moved in a proximal direction against the distal end 700 of the catheter, and twisted so as to engage the interior threads 692 with the exterior threads 712 on the catheter. Longitudinal translation of the catheter relative to the catheter retainer 600 may thus be prevented, and the catheter will thus be releasably retained and the fluid tight seal maintained.

Although not illustrated in the present embodiment, the tubular member 680 may comprise a radially extending member located near the distal end of the tubular member 680, which is configured to engage a corresponding receptacle on the distal side of the spin nut 690. The radially extending member may permit the spin nut 690 to be releasably secured in place when no catheter is attached, or during attachment or detachment of the catheter. In embodiments in which the radially extending member inhibits rotation of the spin nut when the radially extending member engages the spin nut 690, disengagement of the catheter from the tubular body 680 may be facilitated by the use of the secured spin nut as a grip by the medical personnel to transfer force to the tubular body 680.

In alternate embodiments, not shown, it will be understood that rather than including two or more bodies extending from the catheter retainer so as to house the two or more lumens extending distally from the catheter retainer, the catheter retainer may include a multi-lumen structure for at least a portion of the length of the lumens. This multi-lumen structure may take the form of concentric tubular, or otherwise hollow, bodies sharing a common longitudinal axis, such that the tubular body defining the inner lumen extends through at least a portion of the second lumen. The outer tubular body may thus serve as a sheath enclosing the inner tubular body. In still other embodiments, the multi-lumen structure may take other forms, such as a single tubular structure comprising a divider defining side-by-side lumens. It will be understood that the above examples of multi-lumen structures are merely exemplary, and that any suitable multi-lumen structure may be utilized.

The various embodiments of the components of the modular catheterization system fitting described above in accordance with the present invention thus provide a means to provide a second fluid connection with an indwelling catheter without disturbing the first fluid connection. In addition, the techniques described may be broadly applied for use with a variety of medical lines and medical procedures.

Of course, it is to be understood that not necessarily all such objects or advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other objects or advantages as may be taught or suggested herein.

Furthermore, the skilled artisan will recognize the interchangeability of various features from different embodiments. For example, a component housing having any suitable retaining space to retain a desired component may be utilized. In addition, in the assembly of FIG. 19, an integral probe without the need for a separate component retainer may be provided, e.g., in place of the protective cap. In other embodiments, probes other than optrode probes or in addition to optrode probes may be provided. In further embodiments, more than one access port may be provided through the inclusion of additional tubular bodies extending from the central portion of the catheter retainer. In addition to the variations described herein, other known equivalents for each feature can be mixed and matched by one of ordinary skill in this art to construct releasable modular catheterization systems in accordance with principles of the present invention.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. In particular, while the present system has been described in the context of particularly preferred embodiments, the skilled artisan will appreciate, in view of the present disclosure, that certain advantages, features and aspects of the anchor may be realized in a variety of other applications, many of which have been noted above. For example, while particularly useful for small-scale applications, such as the illustrated medical application, the skilled artisan can readily adopt the principles and advantages described herein to a variety of other applications, including larger scale devices.

Additionally, it is contemplated that various aspects and features of the invention described can be practiced separately, combined together, or substituted for one another, and that a variety of combination and subcombinations of the features and aspects can be made and still fall within the scope of the invention. Thus, it is intended that the scope of the present invention herein disclosed should not be limited by the particular disclosed embodiments described above, but should be determined only by a fair reading of the claims that follow.

What is claimed is:

1. A modular catheterization system for placing a catheter in parallel flow communication with a medical line and a sensor, the system comprising:
a first anchor pad;
a housing being supported by the first anchor pad and having a receiving area configured to receive at least a portion of the sensor;
a second anchor pad; and
a retainer being supported by the second anchor pad and having a central body, a first tubular body, and a second tubular body, the central body having a proximal end configured to receive the catheter in a fluid-tight manner, the first tubular body and the central body forming a first luminal path between the catheter and the medical line, and the second tubular body and the central body forming a second luminal path between the catheter and the sensor.

2. The system of claim 1 additionally comprising a movable member configured to occlude fluid flow through the second luminal path when the movable member is in a first position, and to permit fluid flow through the second luminal path when the movable member is in a second position.

3. The system of claim 2, wherein the movable member is biased to remain in the first position, the movable member being movable in a proximal direction to said second position.

4. The system of claim 2, wherein the second tubular body extends in a direction substantially parallel to a longitudinal axis of the catheter housing.

5. The system of claim 4, wherein the first tubular body is oriented at an angle to the second tubular body.

6. The system of claim 1 further comprising two resilient retention members extending transversely upward from a base of the retainer, the retention members comprising upper portions which curve laterally inward so as to inhibit upward translation in a transverse direction of a retained portion of the catheter.

7. The system of claim 1 further comprising a proximally extending clip member having an overhanging proximal end which extends transversely downward, said proximal end having a distal surface being configured to abut a proximal surface of a radially extending portion of the catheter so as to inhibit longitudinal translation of the catheter in at least a proximal direction.

8. The system of claim 1 further comprising a proximally extending tubular body and a spin nut disposed on said body, said spin nut being longitudinally and rotationally movable relative to the proximally extending tubular body, wherein said spin nut comprises a threaded interior surface configured to engage the catheter.

9. The system of claim 1 further comprising a proximally extending tubular body, wherein said proximally extending tubular body is configured to be inserted into and retained within a corresponding opening in the distal portion of the retained catheter so as to form a fluid tight seal between the catheter and the catheter retainer.

10. A catheterization system for co-locating a sensor and a catheter in close proximity on a patient, the system comprising:
a first anchor;
a first retainer being supported by the first anchor and having a receptacle, a manifold, a first conduit, and a second conduit, the receptacle being configured to receive at least a portion of the catheter, the first and second conduits being in fluidic communication with the manifold;
a second anchor having a size and a shape so as to be located proximate to the first anchor; and
a second retainer being supported by the second anchor and having a receiving area configured to receive at least a portion of the sensor.

11. The system of claim 10 additionally comprising an occluding member disposed within the second conduit so as to occlude fluid flow through the second conduit.

12. The system of claim 11, wherein the occluding member is movable via a proximally extending portion of the sensor so as to permit fluid flow through the second conduit.

13. The system of claim 10 additionally comprising a pair of resilient members located on either side of the receptacle, each resilient member having an upper portion curving laterally towards the center of the receptacle, the resilient members being deformable away from the receptacle to permit a portion of the catheter to be inserted therebetween, wherein upward translation in a transverse direction is inhibited by the upper portions of the resilient members.

14. The system of claim 10 additionally comprising a proximally extending body, the proximally extending body comprising a distal tip configured to engage an opening in the distal end of the catheter.

15. The system of claim 14 additionally comprising a rotatable member disposed on said proximally extending body, said rotatable member comprising an interior surface having retention features configured to engage the distal end of the catheter.

16. A modular catheterization system for allowing vascular access via an existing indwelling device, the system comprising:
a sensor housing disposed on a first anchor pad, wherein the sensor housing comprises a channel configured to retain a portion of a sensor;
a catheter retainer disposed on a second anchor pad, said catheter retainer comprising:
a central body comprising a first end, wherein the first end is configured to be secured to and placed in fluid communication with a distal end of a catheter via an aperture extending through said first end;
a first tubular body extending outward from said central body and comprising a second aperture, wherein a first luminal path extends through the first tubular body from the second aperture to the first aperture;
a second tubular body extending outward from said central body and comprising a third aperture, wherein a second luminal path extends through the second tubular body from the third aperture to the first aperture; and
an obstructing member positioned along the second luminal path so as to inhibit fluid flow along the second luminal path without inhibiting fluid flow along the first luminal path; and
a sensor comprising a body portion configured to be retained within the sensor housing, and an outwardly extending member having a proximal end configured to enter said third aperture and interact with the obstructing member to permit passage of said proximal end beyond the obstructing member;
said first and second anchor pads being configured to permit co-location of the sensor housing and catheter retainer.

17. The system of claim 16 further comprising a movable flap biased to move in a distal direction.

18. The system of claim 16 further comprising a piercable septum.

19. The system of claim 16 further comprising an optrode probe.

20. The system of claim 16 further comprising a retention surface configured to abut a surface of the sensor to inhibit longitudinal translation of the sensor in a distal direction relative to the sensor housing.
